(19) United States
(12) Patent Application Publication
(10) Pub. No.: US 2009/0088769 A1
(43) Pub. Date: Apr. 2, 2009

(54) Spinal Fixation Alignment Apparatus

(76) Inventor: Steven C. Poletti, Sullivan's Island, SC (US)

Correspondence Address: KENYON & KENYON LLP, 1500 K STREET N.W., SUITE 700, WASHINGTON, DC, 20005, US

(21) Appl. No.: 12/211,533

(22) Filed: Sep. 16, 2008

Related U.S. Application Data
(60) Provisional application No. 60/960,443, filed on Sep. 28, 2007.

(51) Int. Cl. A61B 17/58; A61B 17/04

(52) U.S. Cl. 606/103; 606/301; 606/305; 606/308

(57) ABSTRACT

Alignment apparatuses, methods, and systems for positioning guidewire insertion devices into a patient's vertebrae. The alignment apparatus can facilitate insertion of a guidewire insertion device through a stab incision made in a patient's skin and into target vertebrae of the patient in a spinal fixation procedure. The apparatus may comprise a housing having at least one opening. The housing can be positionable against the patient's skin proximate to the target vertebrae. The opening may be configured to receive the guidewire insertion device so that it directs the advancement of the guidewire insertion device through the opening and the stab incision.

### TECHNICAL FIELD

Thee present application relates to a spinal fixation alignment apparatus.

### BACKGROUND

The human spinal column consists of a series of thirty-three stacked vertebrae. Each vertebra is separated by a disc and includes a vertebral body having several posterior facing structures. Through injury or disease, one or more of these components of a vertebra become damaged thereby necessitating spinal procedures to remove or modify the damaged component. One way to stabilize the spinal column after such procedures is through spinal fixation.

One way to stabilize the spinal column, after removal of facet joints or other posterior elements of the spine, is through a spinal fixation procedure. A spinal fixation procedure can involve rigidly or dynamically fixing adjacent stacked vertebrae vertically through bone grafting and/or rigid mechanical fixation assemblies. In each case, the adjacent vertebrae may be fixed to one another through a medical procedure. A spinal fixation device used in such a procedure may be a rigid or semi-rigid mechanical support system which is surgically implanted into the vertebral column in order to obtain stabilization of spinal fractures, correction of spinal deformities, or treatment of degenerative spinal disease. The spinal fixation device may be comprised of rods, plates, screws and/or interbody constructs such that medical personnel can obtain optimum fixation in each case. The spinal fixation device can be made of a rigid or semi-rigid biologically compatible material, such as titanium alloy.

For example, FIG. 1a shows a portion of a spinal fixation device 2 utilizing pedicles screws 4a-c and rods 6 to fix the L2, L3, and L4 vertebrae of a patient together. Since more than two vertebrae are being fixed to one another, the illustrated procedure is referred to as a multi-level spinal fixation procedure.

Turning to FIG. 2, an enlarged view of a pedicle screw 4 is illustrated. As can be seen, an opening 8 may be provided on an upper end of the pedicle screw 4. This opening 8 is configured to receive a portion of the rod 6 which, as seen in the example of FIG. 1a, may extend through openings of three pedicle screws 4.

During implantation of the spinal fixation device, medical personnel can maneuver rod 6 through respective openings 8 of the pedicle screws 4 using x-ray guidance and/or imaging. Once the rod 6 is positioned in the openings 8, the rod 6 may be secured within the openings 8 by using any means well known in the art (e.g., clamp screw).

Even though positioning of the rod 6 within the openings 8 of the multiple pedicle screws 4 may be possible using x-ray guidance and/or imaging, positioning can become increasingly difficult in multi-level spinal fixation procedures where three or more pedicle screws are used. For instance, as seen in FIG. 1b, if one of the pedicle screws 4 is not aligned with an adjacent pedicle screw 4, passing a rigid and linear rod 6 through three or more openings 8 can be difficult, or in some instances impossible. This may be one reason that percutaneous multi-level spinal fixation procedures are seldom performed.

### SUMMARY

The present application is directed to apparatuses, systems, and methods for positioning guidewire insertion devices into a patient's vertebrae in a spinal fixation procedure.

In an embodiment, the present invention provides an alignment apparatus for positioning a guidewire insertion device in a spinal fixation procedure. The apparatus comprises a housing defining a horizontal plane and having a plurality of openings, each of the plurality of openings having a center, and a central axis extending through the center. Each central axis is parallel to each other central axis and all central axes lie in an angled plane. The angled plane defines an angle between the horizontal plane of the housing and the angled plane. The apparatus further comprises at least one guidewire insertion device removably received within the plurality of openings.

The invention may be embodied by numerous other apparatuses, systems, and methods. The description provided herein, when taken in conjunction with the annexed drawings, discloses examples of the invention. Other embodiments, which incorporate some or all steps as taught herein, are also possible.

### BRIEF DESCRIPTION OF THE DRAWINGS

The present invention will become more filly understood from the detailed description given hereinbelow and the accompanying drawings which are given by way of illustration only, and thus are not limitative of the present invention. Referring to the drawings, which form a part of this disclosure:

FIG. 1a shows a portion of a conventional fixation device implanted within a patient and FIG. 1b shows a similar portion of a spinal fixation device having misaligned pedicle screws;

FIG. 2 shows a side view of a conventional pedicle screw utilized in the fixation device of FIG. 1a;

FIG. 3a shows an elevational view of an alignment apparatus as maybe employed in accordance with certain embodiments of the present invention;

FIG. 3b shows an alternative arrangement of the alignment apparatus of FIG. 3a as may be employed in accordance with certain embodiments of the present invention;

FIGS. 4 shows an elevational view of a stab incision being made in a patient's skin in accordance with certain embodiments of the present invention;

FIGS. 5a-d show the alignment apparatuses of FIGS. 3a-b being used to insert guidewire insertion devices into a patient's vertebrae;

FIG. 6 shows a plurality of guidewires positioned in a patient's vertebrae; and

FIG. 7 is a flow chart of method steps that may be employed in accordance with certain embodiments of the present invention.

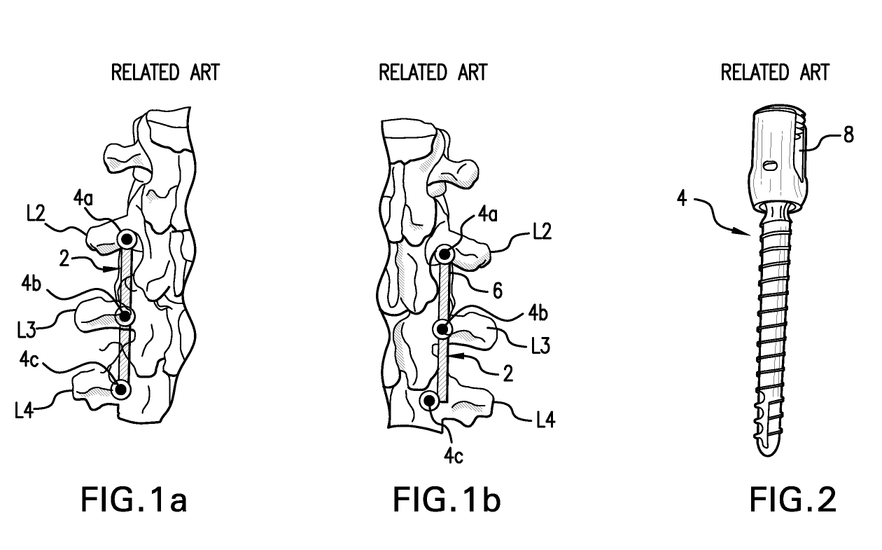

### DETAILED DESCRIPTION

Embodiments of the present invention are directed to spinal fixation alignment apparatuses, systems, and methods for positioning a guidewire insertion device(s) into a patient's vertebrae, thus resulting in proper alignment of subsequently positioned guidewire(s) and pedicle screw(s).

In accordance with certain embodiments of the present invention, an alignment apparatus is provided to facilitate vertical alignment of pedicle screws, and their openings, for performing multi-level spinal fixation procedures percutaneously. More specifically, in order to facilitate alignment of a guidewire insertion device(s), thus resulting in proper alignment of subsequently positioned guidewire(s) and pedicle screws, certain embodiments of the present invention regard an alignment apparatus that may be used in a multi-level spinal fixation procedure.

In a typical spinal fixation procedure, a guidewire insertion device (such as, for example, large bore needles) may be positioned within a vertebra. Then a guidewire can be positioned in the vertebra through a passageway of the guidewire insertion device. A pedicle screw may then be positioned in the vertebra via advancement over the guidewire. The pedicle screw may be self-tapping and/or may be engaged into predrilled holes formed by the guidewire insertion device. For a multi-level spinal fixation procedure, these steps are also involved for each adjacent vertebra. As stated herein above, proper alignment of a guidewire insertion device may result in proper alignment of the guidewires and pedicle screws. For example, in a multi-level spinal fixation procedure for the L2, L3, and L4 vertebrae, three pedical screw openings should be aligned in the same vertical (sagittal) plane.

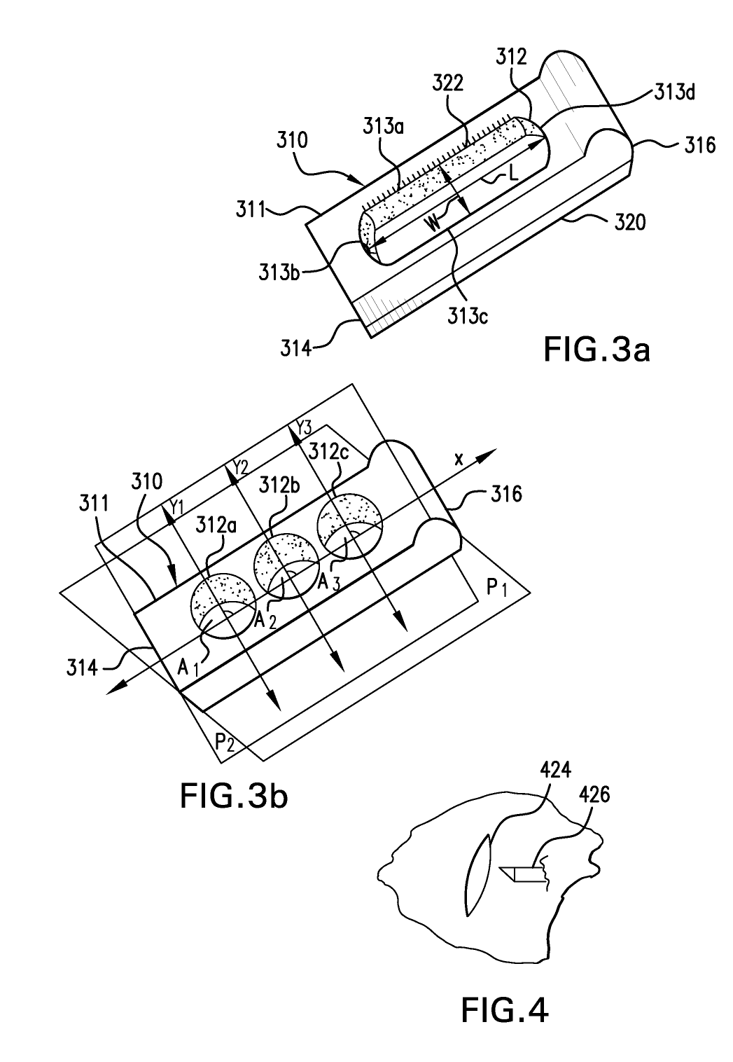

Referring to FIG. 3a, an alignment apparatus 310 that may be employed in accordance with certain embodiments of the present invention is shown. In the example, the alignment apparatus 310 includes a housing 311 having a centrally disposed elongated opening 312 which limits movement, such as lateral and rotational movement, of the guidewire insertion device while being located therein. Although in this embodiment the opening 312 extends along the length L of the housing 311 other arrangements may be used and the opening may not be centrally disposed. In more detail, in this embodiment, an alignment apparatus comprises a housing having an opening which is defined by four walls 313a, 313b, 313c, and 313d. As illustrated in FIG. 3a, two of the walls, 313a and 31 3c, lie along the length L of housing 311 and are straight and parallel to each other. In certain embodiments, the length L of the opening is greater than the width W of the opening as illustrated in FIG. 3a. Still in certain embodiments, the width W of the opening is approximately the size of a diameter of a guidewire insertion device to limit lateral and rotational movement of the guidewire insertion device.

FIG. 3b shows an alternative arrangement for an alignment apparatus. The apparatus of FIG. 3b is similar to that of FIG. 3a, however a plurality of openings are provided in place of a single elongated opening. Specifically, in this embodiment, the alignment apparatus comprises a housing defining a horizontal plane P₁ and having a plurality of openings 312a, 312b, and 312c. Each of the plurality of openings has a center and a central axis Y extending through the center. Each central axis Y₁, Y₂, and Y₃ is parallel to each other central axis and all central axes lie in an angled plane P₂. As illustrated in FIG. 3b, there is an angle A defined between axis X, which lies in horizontal plane P₁ of the housing, and the angled plane P₂. In preferred embodiments, angle A is 90° but other suitable angles could also be defined. In this example, three openings 312a, 312b, and 312c are shown; however, any number of openings may be used. The number of openings may, for example, be equal to the number of vertebrae being fixed together. For instance, the alignment apparatus 310 of FIG. 3b can be used to fix three vertebrae, such as the L2, L3, and L4 vertebrae in a multi-level spinal fixation procedure.

The opening(s) of the housing may be any suitable size and shape. For example, in certain embodiments of the present invention, for instance, where a large bore needle is used as the guidewire insertion device and the housing defines a single opening, the opening may have a width (W) of about 0.075 to 0.15 inches. In embodiments, where multiple openings are used. The opening(s) may be sized slightly larger than the diameter and/or width of the guidewire insertion device to frictionally retain the needle and prevent movement (e.g., lateral and rotational movement) within the opening(s). In addition, the opening(s) may be provided with serrations, indentations, and/or protrusions to prevent lateral movement of the guidewire insertion device within the opening.

Further, the elongated opening may be any suitable length. For example, when the alignment apparatus is being used in a multi-level spinal fixation procedure, the opening 312 may be at least 4.50 inches long. This distance is equal to the minimum length between the L2, L3, and L4 vertebrae on a typical patient. The housing 311 can be positioned externally against the skin of a patient. For example, in a spinal fixation procedure, the housing 311 may be positioned externally against the skin and proximate to one or more target vertebrae.

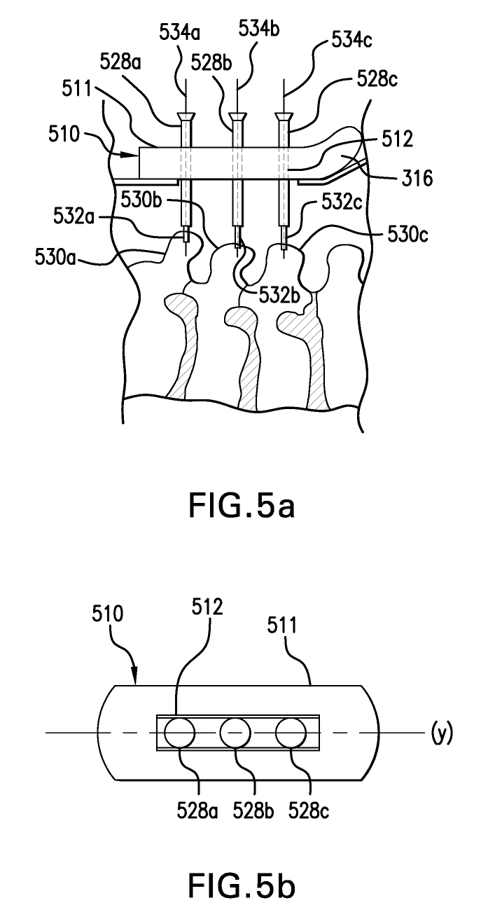

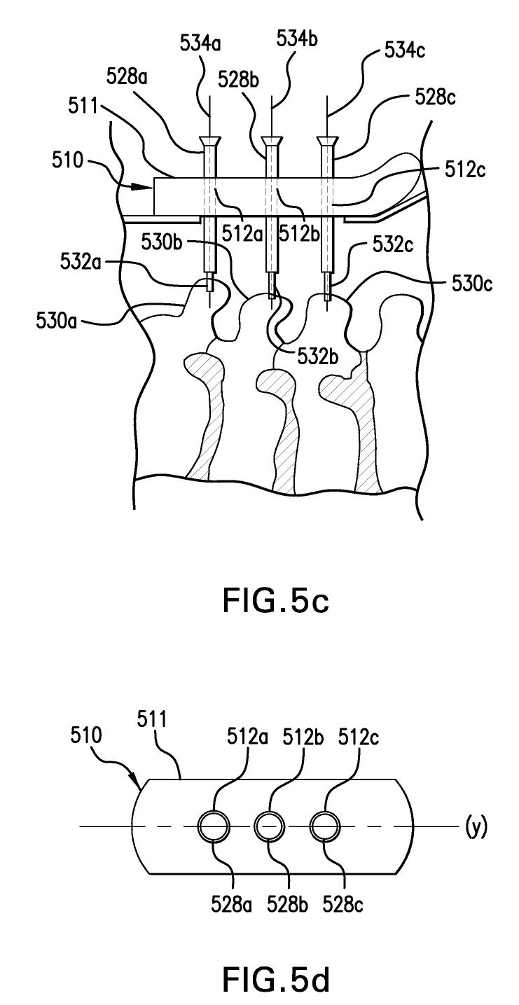

Since the housing 311 may be positioned against the patient's skin, in certain embodiments of the present invention, the housing 311 may be shaped to conform to the shape of the patient's back (FIG. 5a). For example, as seen in FIG. 5a, an end 314 and/or 316 of the housing 311 can be curved so as to conform to the patient's lower back. Likewise, to prevent movement of the housing 311 with respect to the patient's skin, an adhesive may be used. For example, as seen in FIG. 3a, an adhesive layer 320 is positioned on the bottom surface 318 of the housing 311.

To facilitate insertion of a guidewire insertion device, the housing 311 may be formed of a radiolucent material. Any material which allows X-rays to pass therethrough may be used. For example, hard plastics may be suitable.

Markings 322 may also be placed on surfaces of the alignment apparatus to facilitate alignment of the guidewire insertion device within the slot and/or into a vertebra. For example, as seen in FIG. 3a, indicia 312 (e.g., units of length such as millimeters) can be placed on a top surface of the housing 311; however, other arrangements may be used.

The present also provides systems for positioning a guidewire into a patient's vertebrae in a spinal fixation procedure including an alignment apparatus according to embodiments described herein or other embodiments and an guidewire insertion device adapted to be removable received within the an opening or each of a plurality of openings of an alignment apparatus.

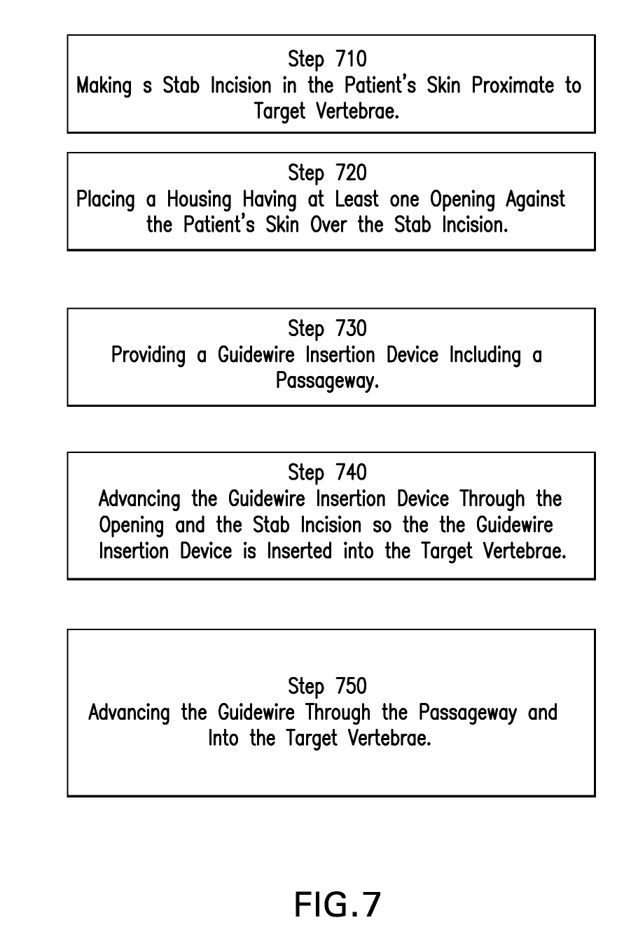

FIG. 7 shows a flow chart including method steps that may be employed with certain embodiments of the present application for positioning a guidewire into a patient's vertebra in a spinal fixation procedure. Step 710 may include making a stab incision in the patient's skin proximate to a target vertebra. Step 720 may include placing a housing having at least one opening against the patient's skin over the stab incision. Step 730 can include providing a guidewire insertion device including a passageway. Step 740 can include advancing the guidewire insertion device through the at least one opening of the housing and the stab incision so that the guidewire insertion device is inserted into the target vertebra. Step 750 may include advancing the guidewire through the passageway and into the target vertebra. Subsequent steps can include providing a second guidewire insertion device and positioning the second guidewire insertion device within the housing.

The present invention also provides a method for positioning pedicle screws into a patient's vertebrae which include the steps identified in FIG. 7 and include the additional step of passing pedicle screws over the guidewire and introducing the pedicle screws into a target vertebrae.

FIG. 4 shows an elevational view of a stab incision 424 being made in a patient's skin in accordance with certain embodiments of the present invention. The stab incision 424 may be formed in the patient using any suitable means which are well known in the art. In the example, a knife 426 is being used.

FIGS. 5a-d show an alignment apparatuses 510 similar to the alignment apparatuses of FIGS. 3a -b being used to insert guidewire insertion devices 528a, b, c into a patient's vertebrae 530a, b, c.

FIGS. 5a-b shows three guidewire insertion devices 528a, b, c positioned within three vertebrae 530a, b, c in a multi-level spinal fixation procedure. In the example, the guidewire insertion devices 528a, b, c are large bore needles; however, other devices well known in the art may be used. The needles 532a, b, c of the guidewire insertion devices 528a, b, c may be inserted into the vertebrae 530a, b, c through an elongated slot 512 on the housing 511 of the alignment apparatus 510.

FIG. 5b shows that the elongate opening 512 can be used to align the guidewire insertion devices 528a, b, c along the same vertical plane (e.g., sagittal plane), which extends along line (y) and perpendicular into the paper in this example.

FIGS. 5c-d are similar to those of FIGS. 5a-b, except that three openings 512a, b, c are used instead of the elongated slot of FIGS. 5a, b. As seen in FIG. 5d, as with the single opening, the three openings 512a, b, c can be used to align the guidewire insertion devices 528a, b, c in the same vertical plane.

In the examples of FIGS. 5a-d, once the guide wire insertion devices 528a, b, c are positioned in the same plane, guidewires 534a, b, c may be advanced through passageways of the guidewire insertion devices 528a, b, c and into the target vertebrae 530a, b, c. Once the guidewires 534a, b, c are properly positioned, the guidewire insertion devices 528a, b, c and alignment apparatus 510 maybe removed from the patient and the pedicle screws can be placed over the guidewires and introduced into the target vertebrae.

In the examples of FIGS. 5a-d, a separate guidewire insertion device is provided for each guidewire. For example, in FIGS. 5a-b, three guidewire insertion devices are provided within the at least one opening. Similarly, in FIGS. 5c-d, a separate guidewire insertion device is provided for each of the openings.

In accordance with other embodiments of the present application, a single guidewire insertion device may be repositioned within the at least one opening of the housing for insertion of each guidewire. For example, with respect to FIGS. 5c-d, a single guidewire insertion device may be repositioned within each opening for insertion of a respective guidewire. Further in other embodiments, a single guidewire may be repositioned within the at least one opening of the housing for passage of a pedicle screw over the guidewire. For example, with respect to FIGS. 5c-d, a single guidewire may be positioned within the at least one opening and after passage of a pedicle screw, repositioned within the at least one opening for passage of another pedicle screw.

In other embodiments, still other arrangements may be used.

In certain embodiments of the present application, as discussed briefly herein above, the guidewire insertion devices 528a, b, c may be inserted using the assistance of x-ray guidance and/or imaging, such as fluoroscopy, as is well known in the art. For example, medical personnel may consult real-time images of the cavity proximate the target vertebrae of a patient through the use of a fluoroscope. The fluoroscope may comprise x-ray sources and a fluorescent screen between which a patient is placed. In addition, x-ray image intensifiers, CCD video and cameras allowing the images to be played and recorded on a monitor, and flat-panel detector systems which reduce the radiation dose to the patient may also be used.

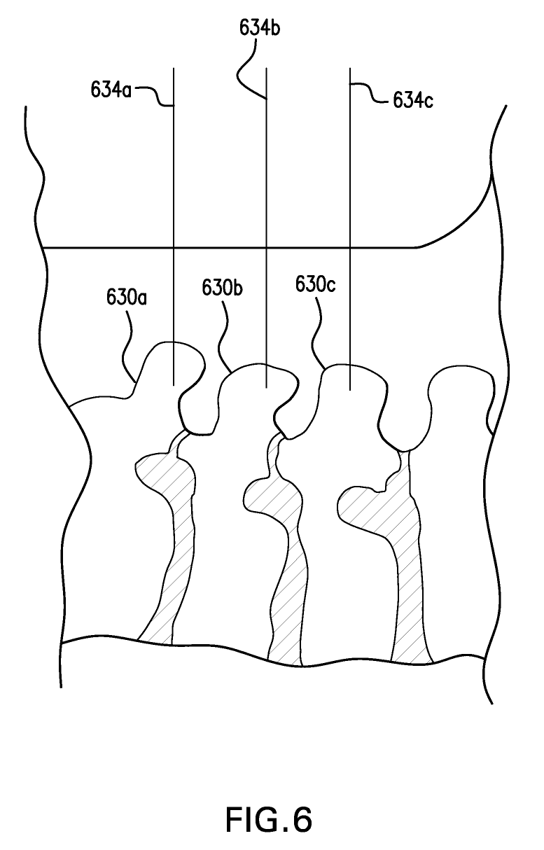

FIG. 6 shows a plurality of guidewires 634a, b, c positioned within a patient's vertebrae 630a, b, c. The guidewires 630a, b, c, in this example, are ready to receive a plurality of pedicle screws thereon. Any guidewire that is well known in the art may be used. For example, a thin and flexible wire, that can be inserted into a confined or tortuous space, configured to receive a stiffer bulkier instrument thereon is suitable.

In embodiments, not shown, the sequence of method steps may be reordered and steps may be added or removed. The steps may also be modified.

While various embodiments have been described, other embodiments are possible. It should be understood that the foregoing descriptions of various examples of the alignment apparatus are not intended to be limiting, and any number of modifications, combinations, and alternatives of the examples may be employed to facilitate implantation of a spinal fixation device.

The examples described herein are merely illustrative, as numerous other embodiments may be implemented without departing from the spirit and scope of the exemplary embodiments of the present application. Moreover, while certain features of the application may be shown on only certain embodiments or configurations, these features may be exchanged, added, and removed from and between the various embodiments or configurations while remaining within the scope of the application. Likewise, methods described and disclosed may also be performed in various sequences, with some or all of the disclosed steps being performed in a different order than described while still remaining within the spirit and scope of the present application.

1. A system for positioning a guidewire into a patient's vertebrae in a spinal fixation procedure, the system comprising:
an alignment apparatus for positioning a guidewire insertion device comprising:
a housing defining a horizontal plane and having a plurality of openings,
each of the plurality of openings having a center, and a central axis extending through the center, each central axis being parallel to each other central axis and all central axes lying in an angled plane, an angle defined between the horizontal plane of the housing and the angled plane; and
at least one guidewire insertion device adapted to be removably received within each of the plurality of openings.

2. The alignment apparatus of claim 1, wherein the angle is 90°.

3. The alignment apparatus of claim 1 wherein the plurality of openings comprises three or more openings.

4. The alignment apparatus of claim 1, wherein the housing is radiolucent and is adapted to be positioned against an external surface of a patient's skin.

5. The alignment apparatus of claim 1, wherein each of the plurality of openings is between about 0.075 and 0.15 inches wide.

6. The alignment apparatus of claim 1, wherein the housing includes an adhesive to prevent movement of the housing with respect to a patient's skin.

7. A system for positioning a guidewire into a patient's vertebrae in a spinal fixation procedure, the system comprising:
an alignment apparatus for positioning a guidewire insertion device comprising:
a housing having an opening, the opening being defined by four walls,
wherein two of said four walls lie along a length of the housing, and are straight and parallel to each other; and
at least one guidewire insertion device adapted for being removably received within said opening.

8. The alignment apparatus of claim 7, wherein the length of the opening is greater than the width of the opening.

9. An alignment apparatus for positioning a guidewire insertion device for spinal surgery comprising:
a housing defining an opening,
said opening having a length and a width,
the length being greater than the width, and
the width being approximately the size of a diameter of a guidewire insertion device.

10. A method for positioning a guidewire in a patient's vertebrae in a spinal fixation procedure, comprising the steps of:
making an incision in the patient's skin proximate to a target vertebra;
placing a housing having at least one opening against the patient's skin over the incision;
providing a guidewire insertion device having a passageway;
advancing the guidewire insertion device through the at least one opening and the incision, thereby inserting guidewire insertion device into the target vertebra; and
advancing a guidewire through the passageway and into the target vertebra.

11. The method of claim 10, further comprising:
removing the guidewire insertion device after the guidewire is advanced.

12. The method of claim 10, wherein the at least one opening is elongated along a length of the housing.

13. The method of claim 10, wherein the at least one opening comprises three openings.

14. The method of claim 10, further comprising:
positioning the housing against an external surface of the patient's skin.

15. The method of claim 10, further comprising:
re-positioning the guidewire insertion device within the opening to insert a second guidewire into a second vertebra.

16. The method of claim 10, wherein the at least one opening further comprises a second opening, further comprising the step of:
re-positioning the guidewire insertion device within the second opening to insert a second guidewire into a second vertebra.

17. The method of claim 10, further comprising:
providing a second guidewire insertion device; and
positioning the second guidewire insertion device with the opening to insert a second guidewire into a second vertebra.

18. The method of claim 10, further comprising:
providing a third guidewire insertion device; and
positioning the third guidewire insertion device within a second opening to insert a third guidewire into a third vertebra.
